(19) United States
(12) Patent Application Publication
(10) Pub. No.: US 2024/0188988 A1
(43) Pub. Date: Jun. 13, 2024

(54) DEVICE FOR FEEDING AND REMOVING SURGICAL MATERIAL

(71) Applicant: KARL STORZ SE & Co. KG, Tuttlingen (DE)

(72) Inventors: Janosz Schneider, Tuttlingen (DE); Jochen Stefan, Tuttlingen (DE)

(73) Assignee: KARL STORZ SE & Co. KG, Tuttlingen (DE)

(21) Appl. No.: 18/530,931

(22) Filed: Dec. 6, 2023

(30) Foreign Application Priority Data
Dec. 8, 2022 (DE) 102022132716.7

(51) Int. Cl. A61B 17/34; A61B 17/00; A61B 34/00

(52) U.S. Cl. CPC A61B 17/3423; A61B 34/25; A61B 2017/00398; A61B 2017/00477

(57) ABSTRACT

The disclosure relates to a device for supplying operating material into an endoscopic operating site or for removing operating material from an endoscopic operating site, comprising a trocar and a medical instrument that is insertable into the operating site through the trocar. According to the disclosure, a device which serves the supply of operating material into an endoscopic operating site or for the removal of operating material from an endoscopic operating site and which, while being easy to handle, allows reliable supply and removal of operating material, is characterized by a loadable magazine which is arranged at the proximal end of the trocar and serves to accommodate material containers which, by means of the medical instrument and through the trocar, are insertable into the operating site or removable therefrom.

### TECHNICAL FIELD

The disclosure relates to a device for supplying operating material into an endoscopic operating site or for removing operating material from an endoscopic operating site, comprising a trocar and a medical instrument that is insertable into the operating site through the trocar.

### BACKGROUND

It is frequently necessary to introduce operating material, for example suturing material, into the operating site and remove operating material, for example tissue samples, from the operating site during an endoscopic operation. In practice, the supply and removal of operating material is implemented by means of a medical instrument, for example a pair of forceps, which is introduced into the operating site and removed therefrom through a trocar.

By all means, this procedure for introducing and removing operating material has proven its worth in endoscopic surgery: however, depending on the operating material, it requires specific medical instruments to introduce said operating material into the operating site or remove it therefrom, and moreover the use of relatively large trocars may lead to insufflation losses via these trocars. Contamination and/or the loss of the tissue sample may occur when tissue samples are removed from the operating site.

### SUMMARY

Using this as a starting point, the disclosure is based on the object of developing a device which serves the supply of operating material into an endoscopic operating site or for the removal of operating material from an endoscopic operating site and which, while being easy to handle, allows reliable supply and removal of operating material.

According to the disclosure, the solution to this problem is characterized by a loadable magazine which is arranged at the proximal end of the trocar and serves to accommodate material containers which, by means of the medical instrument and through the trocar, are insertable into the operating site or removable therefrom.

By means of the device according to the disclosure, it is possible to reliably and easily supply very different operating materials to the operating site or remove these therefrom using only one medical instrument. The material containers are equipped with the appropriate operating material outside of the patient's body and then introduced into the operating site through the trocar by means of a medical instrument. Likewise, material containers introduced into the operating site empty can be used to remove tissue samples, for example, from the operating site through the trocar by means of the medical instrument.

To design the device to be particularly flexible, the material containers are stored in a magazine arranged at the proximal end of the trocar so that empty material containers and also material containers equipped with different operating materials are available ready for use at all times and can be supplied to the operating site by means of the medical instrument. Without switching the medical instrument, the device according to the disclosure allows a material container to be returned into the magazine and another material container to be introduced into the operating site at any time.

A preferred embodiment of the disclosure proposes that the magazine is formed as a cylinder that is rotatable about the longitudinal axis of the trocar. The use of such a revolving magazine is particularly space-saving and easy to handle.

An alternative embodiment of the disclosure proposes that the magazine is formed as a slide that is displaceable across the longitudinal axis of the trocar.

According to the disclosure, the magazine for accommodating the material containers is advantageously drivable by motor, whereby a fast and positionally correct assignment of the required material container is ensured.

In order to be able to insert the respective material container into the operating site through the trocar by means of the medical instrument, a practical embodiment of the disclosure proposes that each material container is securable to a distal instrument tip of the medical instrument by means of a latch mechanism. Designing the coupling system as a latch mechanism represents a connection that is simple to fabricate and safe to handle.

To form the latch mechanism, the disclosure proposes that the latch mechanism consists of a latch element arranged at the distal instrument tip of the medical instrument and a corresponding latch receptacle on the material container. By way of the two mutually corresponding components of latch element and latch receptacle, there is reliable coupling between the distal instrument tip of the medical instrument and the material container to be inserted into the operating site.

Furthermore, the disclosure proposes that the latch element arranged at the distal instrument tip of the medical instrument is rotatable about the longitudinal axis of the medical instrument. Rotatability of the latch element facilitates fast and reliable coupling with the latch receptacle of the material container.

An alternative embodiment according to the disclosure for forming the coupling system between the material container on the one hand and the distal instrument tip of the medical instrument on the other hand proposes that each material container is securable to the distal instrument tip of the medical instrument by means of a magnetic coupling system.

To be able in a reliable and contamination-free manner to introduce the operating material into the operating site or remove this therefrom, the disclosure proposes that each material container has a sealable opening for filling and/or emptying the material container. According to the disclosure, the opening of the material container is advantageously opened and closed by means of the medical instrument that is couplable to the material container, with the result that there is no need for a further medical instrument for opening the material container inserted into the operating site.

A preferred embodiment of the disclosure proposes that the distal instrument tip of the medical instrument that is couplable to the material container is able to be angled in relation to the longitudinal axis of the medical instrument by means of at least one joint. As a result of the distal instrument tip of the medical instrument that is able to be equipped with the material container being able to be angled, the operating material situated in the material container can also be accumulated and taken out in small cavities.

In an alternative or in addition to the distal instrument tip of the medical instrument that is couplable to the material container being angled, the disclosure proposes that the distal instrument tip of the medical instrument that is couplable to the material container is designed to be rotatable about the longitudinal axis of the medical instrument. This can further improve the possibility of the positionally accurate supply of the operating material.

A practical embodiment of the disclosure proposes that the device according to the disclosure is couplable to a medical telemanipulation robot.

In an alternative, the disclosure proposes that the device is couplable to manually operable medical instruments.

Further features and advantages of the disclosure emerge from the associated drawings, which illustrate only by way of example two exemplary embodiments of a device according to the disclosure for supplying operating material into an endoscopic operating site, without the disclosure being limited to these exemplary embodiments. It will be appreciated that the features mentioned above and the features yet to be explained below are applicable not only in the respectively specified combination but also in other combinations or on their own, without departing from the scope of the present disclosure.

### BRIEF DESCRIPTION OF THE DRAWINGS

FIG. 1 shows a perspective plan view of a first embodiment of a device according to the disclosure for supplying operating material into an endoscopic operating site or for removing operating material from an endoscopic operating site,

FIG. 2 shows a side view of the representation according to FIG. 1,

FIG. 3 shows a partial longitudinal section through the representation according to FIG. 2,

FIG. 4 shows a magnified view of detail IV according to FIG. 1,

FIG. 5 shows a back view of the representation according to FIG. 4,

FIG. 6 shows two different perspective views of the medical instrument and a material container,

FIG. 7 shows a longitudinal section through the medical instrument and a material container according to FIG. 6, and

FIG. 8 shows a perspective plan view according to FIG. 1, albeit illustrating a second embodiment of a device according to the disclosure.

### DETAILED DESCRIPTION OF PREFERRED EMBODIMENTS

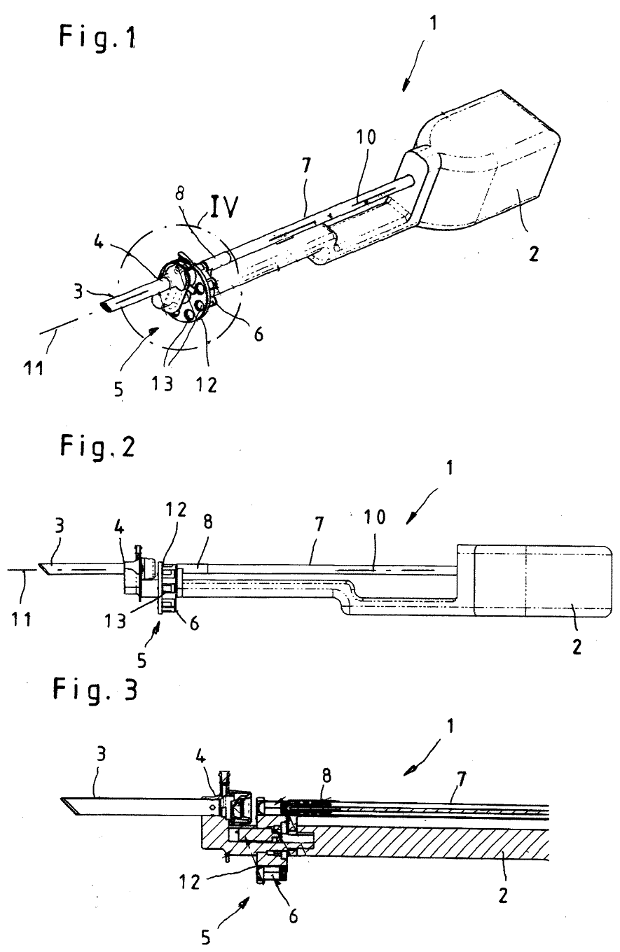

The drawing of FIG. 1 shows a device 1 for supplying operating material into an endoscopic operating site or for removing operating material from an endoscopic operating site. In the embodiment depicted in FIG. 1, the device 1 is coupled to a medical telemanipulation robot 2.

In an alternative, it is naturally also possible to couple the device 1 described in more detail below with further manually operable medical instruments.

The device 1 essentially consists of a trocar 3, a loadable magazine 5 which is arranged at the proximal end 4 of the trocar 3 and serves to accommodate material containers 6, and a medical instrument 7, with the aid of which material containers 6 are insertable into the operating site or removable therefrom through the trocar 3.

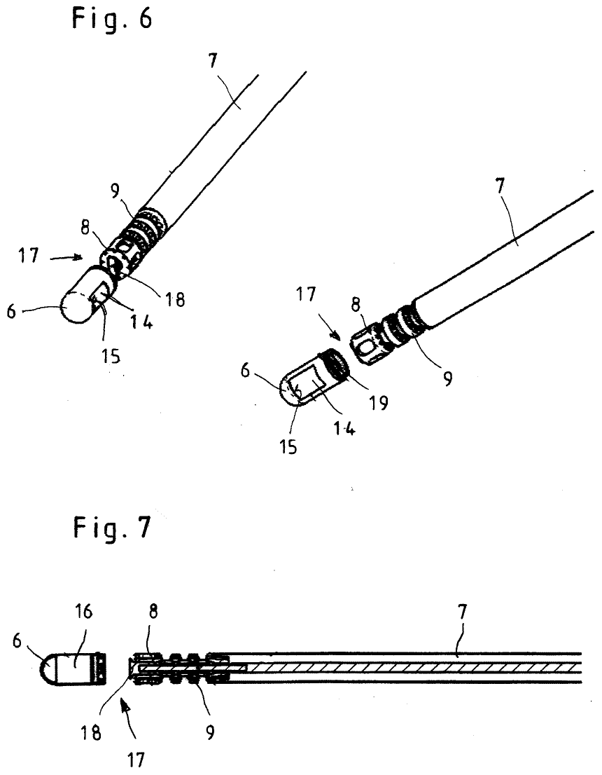

As is evident from the drawings in FIGS. 6 and 7 in particular, the medical instrument 7 comprises a distal instrument tip 8, which is able to be angled in relation to the longitudinal axis 10 of the medical instrument 7 by way of at least one joint 9.

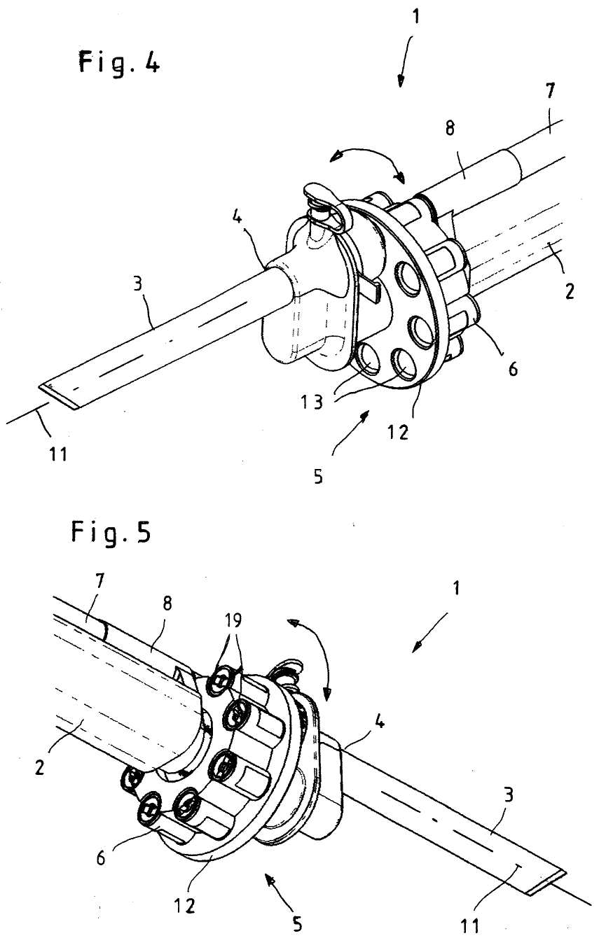

In the first embodiment of the device 1 depicted in the drawings of FIG. 1 to FIG. 5, the magazine 5 which is arranged at the proximal end 4 of the trocar 3 and serves to accommodate the material containers 6 is in the form of a cylinder 12 that is rotatable about the longitudinal axis 11 of the trocar 3, as may be gathered from the arrows in the drawings of FIG. 4 and FIG. 5.

A plurality of accommodation compartments 13 for accommodating a respective material container 6 are formed in the magazine 5 in the form of a cylinder 12. The material containers 6 themselves are in the form of hollow cylindrical capsules, formed in the lateral surfaces of which there is an opening 15 that is closable by way of a flap 14 and allows the container chamber 16 of each material container 6 to be able to be filled and emptied again. Opening and closing the opening 15 by means of the flap 14 is advantageously implemented by way of the medical instrument 7.

Depending on the application or use, the material containers 6 that are insertable into the accommodation compartments 13 of the magazine 5 may already be equipped with necessary operating material, for example needles preassembled with suturing material, or else may be empty, for example if tissue samples are intended to be removed from the operating site to the outside, out of the body of the patient, for the purpose of further examinations. The use of the material containers 6 is advantageous in that material containers 6 equipped at the factory can be loaded into the magazine 5 without contamination. The use of the material container 6 is also advantageous when tissue is taken since the tissue sample can no longer be contaminated and/or lost.

To insert a material container 6 into the operating site through the trocar 3, the distal end of the instrument tip 8 on the one hand and the proximal end of a material container 6 are couplable to one another.

In the embodiment depicted in the drawings of FIG. 5 to FIG. 7, the material container 6 and instrument tip 8 are coupled via a latch mechanism 17 which consists of a latch element 18 arranged at the distal instrument tip 8 of the medical instrument 7 and a corresponding latch receptacle 19 on the material container 6. As evident from the drawing in FIG. 7 in particular, the latch element 18 has a T-shaped embodiment. The latch receptacles 19 of the material containers 6 depicted in FIG. 5 are in the form of slot-shaped openings, into which the T-shaped latch element 18 is insertable.

The latch element 18 arranged at the distal instrument tip 8 of the medical instrument 7 is advantageously rotatable about the longitudinal axis 10 of the medical instrument 7 in order to facilitate quick and reliable coupling with the latch receptacle 19 of the material container 6. Moreover, the rotatability of the latch element 18 can be used to adjust the flap 14 of the material container 6 between a position opening the opening 15 in the material container 6 and a position closing the opening 15.

In an alternative to the coupling between the material containers 6 on the one hand and the distal instrument tip 8 of the medical instrument 7 on the other hand by means of the latch mechanism 17 depicted here, it is also possible for example to provide a magnetic coupling system for the purpose of connecting the two components.

The use of the device 1 is described below on the basis of the drawings in FIG. 1 to FIG. 7.

The drawings in FIGS. 1 and 2 show the device 1 in an initial position, in which the medical instrument 7 is arranged outside of the trocar 3 to the proximal side along a continuation of the trocar 3 and the accommodation compartments 13 of the magazine 5 designed as a cylinder 12 that is rotatable about the longitudinal axis 11 of the trocar 3 are equipped with filled and/or empty material containers 6.

By rotating the cylinder 12 about the longitudinal axis 11 of the trocar 3, the material container 6 required for insertion into the operating site is brought into the coupling position, in which the required material container 6 is arranged in front of the distal instrument tip 8 of the medical instrument 7 along the continuation of the longitudinal axis 11 of the trocar 3.

Subsequently, the medical instrument 7 is displaced in the distal direction toward the trocar 3 in the direction of the longitudinal axis 10 of the medical instrument 7 until the latch element 18 of the instrument tip 8 engages in the latch receptacle 19 of the material container 6 and hence the medical instrument 7 and the material container 6 are coupled to one another via the latch mechanism 17.

To insert the material container 6 into the operating site, the medical instrument 7 is now inserted into the operating site in the body of the patient through the trocar 3 with the instrument tip 8 equipped on the distal side with the material container 6 at the front.

As a result of the distal instrument tip 8 of the medical instrument 7 that is equipped with the material container 6 being able to be angled in relation to the longitudinal axis 10 of the medical instrument 7 by means of the joint 9, the operating material situated in the material container 6 can also be accumulated and taken out in small cavities. Advantageously, the distal instrument tip 8 of the medical instrument 7 equipped with the material container 6 is additionally rotatable about the longitudinal axis 10 of the medical instrument 7 in order to improve the possibility of positionally accurate supply of the operating material even further.

Having arrived at the use point in the operating site, the flap 14 closing the opening 15 of the material container 6 is opened, for example by rotating the latch element 18 about the longitudinal axis 10 of the medical instrument 7, with the result that the operating material stored in the container chamber 16 can be taken out by means of a further medical instrument, which is not depicted here and which was inserted into the operating site via another access, or, in the case of an empty material container 6, the container chamber 16 can be filled with a tissue sample for example.

Once the container chamber 16 of the material container 6 has been emptied or filled in the operating site, the opening 15 of the material container 6 is closed by way of the flap 14 again and the material container 6 is removed from the operating site and the body of the patient again by withdrawing the medical instrument 7 through the trocar 3 in the proximal direction, until the material container 6 is stored in the accommodation compartment 13 of the magazine 5 again.

Subsequently, the latch mechanism 17, by means of which the material container 6 and the instrument tip 8 of the medical instrument 7 are coupled to one another, is released and the medical instrument 7 is pulled further proximally in the direction of its longitudinal axis 10 until the distal-side latch element 18 of the instrument tip 8 has disengaged from the latch receptacle 19 of the material container 6.

If the intention now is to insert another material container 6 into the operating site, then the cylinder 12 of the magazine 5 is rotated about the longitudinal axis 11 of the trocar 3 again until the new material container 6 required for insertion into the operating site is located in the coupling position in which this material container 6 is arranged along the extension of the longitudinal axis 11 of the trocar 3 in front of the distal instrument tip 8 of the medical instrument 7.

The magazine 5 is rotated in motor-driven fashion, especially when the device 1 is coupled to a medical telemanipulation robot 2, whereby fast and precise provision of the respectively required material container 6 is ensured.

By arranging the material containers 6 in a magazine 5 which is able to be equipped with a plurality of material containers 6, it is easily possible to reliably and quickly insert operating material into an operating site or remove this therefrom again.

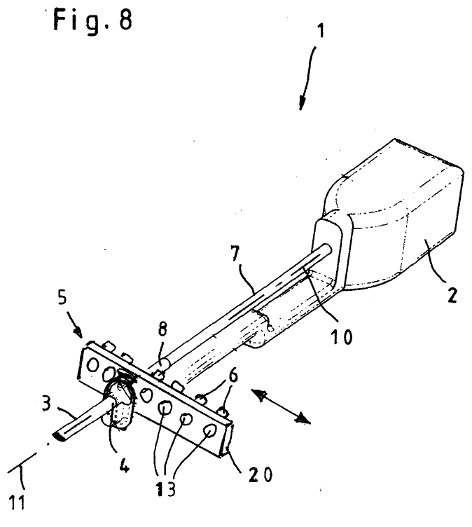

The drawing in FIG. 8 shows an alternative embodiment with regard to the design of the magazine 5 serving to accommodate the material containers 6. As is evident from the drawing, the magazine 5 in this embodiment is designed as a slide 20 that is displaceable in the direction of the arrow across the longitudinal axis 11 of the trocar 3.

Otherwise, the functionality of the device 1 with the magazine 5 in the form of a slide 20 corresponds to the procedure described above on the basis of the device 1 equipped with the rotatable cylinder 12.

An exemplary embodiment of the disclosure is depicted in the drawings. The drawings, the description, and the claims contain numerous features in combination. It will be appreciated that the features mentioned are applicable not only in the respectively specified combination but also in other combinations or on their own, without departing from the scope of the present disclosure. The disclosure relates to a device 1 for supplying operating material into an endoscopic operating site or for removing operating material from an endoscopic operating site, comprising a trocar 3 and a medical instrument that is insertable into the operating site through the trocar 3. According to the disclosure, a device 1 which serves the supply of operating material into an endoscopic operating site or for the removal of operating material from an endoscopic operating site and which, while being easy to handle, allows reliable supply and removal of operating material, is characterized by a loadable magazine 5 which is arranged at the proximal end 4 of the trocar 3 and serves to accommodate material containers 6 which, by means of the medical instrument 7 and through the trocar 3, are insertable into the operating site or removable therefrom.

1. A device for supplying operating material into an endoscopic operating site or for removing operating material from an endoscopic operating site, the device comprising:
a trocar;
a medical instrument that is insertable into the operating site through the trocar; and
a loadable magazine which is arranged at the proximal end of the trocar and serves to accommodate material containers which, by means of the medical instrument and through the trocar, are insertable into the operating site or removable therefrom.

2. The device as set forth in claim 1, wherein the magazine is formed as a cylinder that is rotatable about the longitudinal axis of the trocar.

3. The device as set forth in claim 1, wherein the magazine is formed as a slide that is displaceable across the longitudinal axis of the trocar.

4. The device as set forth in claim 1, wherein the magazine is drivable by motor.

5. The device as set forth in claim 1, wherein each material container is securable to a distal instrument tip of the medical instrument by means of a latch mechanism.

6. The device as set forth in claim 5, wherein the latch mechanism consists of a latch element arranged at the distal instrument tip of the medical instrument and a corresponding latch receptacle on the material container.

7. The device as set forth in claim 6, wherein the latch element arranged at the distal instrument tip of the medical instrument is rotatable about the longitudinal axis of the medical instrument.

8. The device as set forth in claim 1, wherein each material container is securable to the distal instrument tip of the medical instrument by means of a magnetic coupling system.

9. The device as set forth in claim 1, wherein each material container has a sealable opening for filling and/or emptying the material container.

10. The device as set forth in claim 9, wherein the opening of the material container is opened and closed by means of the medical instrument that is configured to couple with the material container.

11. The device as set forth in claim 1, wherein the distal instrument tip of the medical instrument that is configured to couple with the material container is able to be angled in relation to the longitudinal axis of the medical instrument by means of at least one joint.

12. The device as set forth in claim 1, wherein the distal instrument tip of the medical instrument that is couplable to the material container is designed to be rotatable about the longitudinal axis of the medical instrument.

13. The device as set forth in claim 1, wherein the device is couplable to a medical telemanipulation robot.

14. The device as set forth in claim 1, wherein the device is configured to couple with a manually operable medical instrument.
